(19) United States
(12) Patent Application Publication
(10) Pub. No.: US 2009/0259118 A1
(43) Pub. Date: Oct. 15, 2009

(54) Shallow Implantable Analyte Sensor with Rapid Physiological Response

(75) Inventors: Benjamin Jay Feldman, Oakland, CA (US); Udo Hoss, Castro Valley, CA (US); Hyun Cho, Berkeley, CA (US)

Correspondence Address: JACKSON & CO., LLP, 6114 LA SALLE AVENUE, #507, OAKLAND, CA, 94611-2802, US

(73) Assignee: Abbott Diabetes Care Inc., Alameda, CA (US)

(21) Appl. No.: 12/416,126

(22) Filed: Mar. 31, 2009

Related U.S. Application Data
(60) Provisional application No. 61/041,100, filed on Mar. 31, 2008.

(51) Int. Cl. A61B 5/1473

(52) U.S. Cl. 600/345

(57) ABSTRACT

Methods and devices to detect analyte in body fluid are provided. Embodiments include analyte sensors designed so that at least a portion of the sensor is positionable beneath a skin surface in the dermal layer.

### RELATED APPLICATIONS

The present application claims priority under 35 U.S.C. §1 19(e) to U.S. provisional application No. 61/041,100 filed Mar. 31, 2008 entitled “Shallow Implantable Analyte Sensor with Rapid Physiological Response,” and assigned to the assignee of the present application, Abbott Diabetes Care Inc. of Alameda, Calif., the disclosure of which is incorporated herein by reference for all purposes.

### BACKGROUND OF THE DISCLOSURE

The detection of the level of glucose or other analytes, such as lactate, oxygen or the like, in certain individuals is vitally important to their health. For example, the monitoring of glucose is particularly important to individuals with diabetes. Diabetics may need to monitor glucose levels to determine when insulin is needed to reduce glucose levels in their bodies or when additional glucose is needed to raise the level of glucose in their bodies.

Devices have been developed for continuous or automatic monitoring of analytes, such as glucose, in bodily fluid such as in the blood stream or in interstitial fluid. Some of these analyte measuring devices are configured so that at least a portion of the devices are positioned below a skin surface of a user, e.g., in a blood vessel or in the subcutaneous tissue of a user.

Transcutaneous analyte sensors have certain limitations. For example, the insertion of the analyte sensor results in skin/tissue trauma that may result in a period of relative inaccuracy during the initial time period following the sensor insertion exemplified by, for example, false low glucose readings. This may be due to over-consumption of glucose in the positioned sensor vicinity by erthrocytes released by localized bleeding. Further, the analyte sensor response from the positioned sensor in the subcutaneous tissue typically lags the venous glucose primarily due to a physiological lag between subcutaneous and venous glucose.

### SUMMARY

In one aspect, there is provided a method including transcutaneously positioning an analyte sensor through a skin layer of a user, the analyte sensor including an implanted portion having a predetermined length, where said implanted portion of the analyte sensor is positioned within a dermal layer under the skin layer, and detecting a signal generated from the implanted portion of the analyte sensor, wherein the signal is associated with an analyte level of the user.

Also provided are systems and kits.

### BRIEF DESCRIPTION OF THE DRAWINGS

FIG. 1 shows a block diagram of an embodiment of a data monitoring and management system according to the present disclosure;

FIG. 2 shows a block diagram of an embodiment of the data processing unit of the data monitoring and management system of FIG. 1;

FIG. 3 shows a block diagram of an embodiment of a receiver/monitor unit of the data monitoring and management system of FIG. 1;

FIG. 4 shows a schematic diagram of an embodiment of an analyte sensor according to the present disclosure;

FIGS. 5A-5B show a perspective view and a cross sectional view, respectively of an embodiment the analyte sensor of FIG. 4;

FIG. 6 is a graphical illustration of the monitored analyte level with a sensor positioned in the dermal layer compared to a sensor positioned in the interstitial fluid;

FIG. 7 is a graphical illustration of dinner time rise in the analyte sensor response based on the sensor positioned in the dermal layer in comparison to the sensor positioned in the interstitial fluid; and

FIGS. 8A-8C illustrate a perspective view and side cross sectional views of an embodiment of an analyte sensor with microprojection configuration in accordance with one aspect of the present disclosure.

### DETAILED DESCRIPTION

Before the present disclosure is described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Generally, embodiments of the present disclosure relate to methods and devices for detecting at least one analyte such as glucose in body fluid. In certain embodiments, the present disclosure relates to the continuous and/or automatic in vivo monitoring of the level of an analyte using an analyte sensor.

In aspects of the present disclosure, shallow placement of an analyte sensor for positioning the dermal layer under the skin surface of a user is provided. For example, analyte sensors positioned in the dermal layer (typically approximately 0-2 mm under the skin surface, but is to be understood that the location of the dermal layer may vary, e.g., depending on particular anatomy, etc.) in embodiments of the present disclosure result in faster physiological sensor response time compared to the same or similar analyte sensors positioned in the subcutaneous tissue of the same user or subject. In certain embodiments, such sensor placement with the working electrode positioned in the dermal layer has resulted minimal insertion trauma based on reduced insertion depth, and/or also in a more rapid physiological sensor response compared to analyte sensors with the working electrodes positioned in the subcutaneous tissue, yielding an unexpected relative physiological response time.

Aspects include positioning at least the working electrode of an analyte sensor substantially in the dermal layer, for example, at a predetermined angle relative to the skin surface, including substantially parallel to the skin surface. Reference and/or counter electrodes of the analyte sensor may likewise positioned in the dermal layer or positioned elsewhere.

Embodiments include analyte monitoring devices and systems that include an analyte sensor—at least a portion of which is positionable beneath the skin of the user—for the in vivo detection, of an analyte, such as glucose, lactate, and the like, in a body fluid. Embodiments include wholly implantable analyte sensors and analyte sensors in which only a portion of the sensor is positioned under the skin and a portion of the sensor resides above the skin, e.g., for contact to a transmitter, receiver, transceiver, processor, etc. A sensor (and/or a sensor insertion apparatus) may be, for example, be configured to be positionable in a patient for the continuous or periodic monitoring of a level of an analyte in a patient's dermal fluid. For the purposes of this description, continuous monitoring and periodic monitoring will be used interchangeably, unless noted otherwise. The analyte level may be correlated and/or converted to analyte levels in blood or other fluids. In certain embodiments, an analyte sensor may be configured to be positioned in contact with dermal fluid to detect the level of glucose, which detected glucose may be used to infer the glucose level in the patient's bloodstream. For example, analyte sensors may be insertable through the skin layer and into the dermal layer under the skin surface at a depth of approximately 3 mm under the skin surface and containing dermal fluid. Embodiments of the analyte sensors of the subject disclosure may be configured for monitoring the level of the analyte over a time period which may range from minutes, hours, days, weeks, months, or longer.

Of interest are analyte sensors, such as glucose sensors, that are capable of in vivo detection of an analyte for about one hour or more, e.g., about a few hours or more, e.g., about a few days of more, e.g., about three or more days, e.g., about five days or more, e.g., about seven days or more, e.g., about several weeks or at least one month. Future analyte levels may be predicted based on information obtained, e.g., the current analyte level at time, the rate of change of the analyte, etc. Predictive alarms may notify the user of predicted analyte levels that may be of concern prior in advance of the analyte level reaching the future level. This enables the user an opportunity to take corrective action.

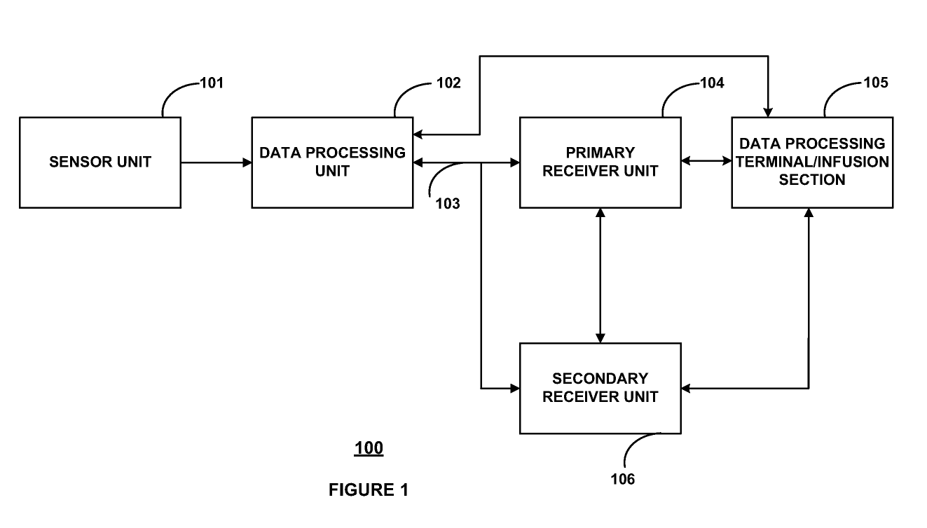

FIG. 1 shows a data monitoring and management system such as, for example, an analyte (e.g., glucose) monitoring system 100 in accordance with certain embodiments. Embodiments of the subject disclosure are further described primarily with respect to glucose monitoring devices and systems, and methods of glucose detection, for convenience only and such description is in no way intended to limit the scope of the disclosure. It is to be understood that the analyte monitoring system may be configured to monitor a variety of analytes at the same time or at different times.

Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

Referring to FIG. 1, the analyte monitoring system 100 includes a sensor 101, a data processing unit 102 including, in one embodiment, a transmitter unit, connectable to the sensor 101, and a primary receiver unit 104 which is configured to communicate with the data processing unit 102 via a communication link 103. In certain embodiments, the primary receiver unit 104 may be further configured to transmit data to a data processing terminal 105 to evaluate or otherwise process or format data received by the primary receiver unit 104. The data processing terminal may be configured to receive data directly from the data processing unit 102 via a communication link which may optionally be configured for bidirectional communication. Further, the data processing unit 102 may include a transmitter or a transceiver to transmit and/or receive data to and/or from the primary receiver unit 104, and/or the data processing terminal 105 and/or optionally the secondary receiver unit 106.

Also shown in FIG. 1 is an optional secondary receiver unit 106 which is operatively coupled to the communication link and configured to receive data transmitted from the data processing unit 102. The secondary receiver unit 106 may be configured to communicate with the primary receiver unit 104, as well as the data processing terminal 105. The secondary receiver unit 106 may be configured for bi-directional wireless communication with each of the primary receiver unit 104 and the data processing terminal 105. As discussed in further detail below, in certain embodiments the secondary receiver unit 106 may be a de-featured receiver as compared to the primary receiver, i.e., the secondary receiver may include a limited or minimal number of functions and features as compared with the primary receiver unit 104. As such, the secondary receiver unit 106 may include a smaller (in one or more, including all, dimensions), compact housing or embodied in a device such as a wrist watch, arm band, etc., for example. Alternatively, the secondary receiver unit 106 may be configured with the same or substantially similar functions and features as the primary receiver unit 104. The secondary receiver may include a docking portion to be mated with a docking cradle unit for placement by, e.g., the bedside for night time monitoring, and/or bi-directional communication device. A docking cradle may recharge a power supply.

Only one sensor 101, data processing unit 102, communication link 103, and data processing terminal 105 are shown in the embodiment of the analyte monitoring system 100 illustrated in FIG. 1. However, it will be appreciated by one of ordinary skill in the art that the analyte monitoring system 100 may include more than one sensor 101 and/or more than one data processing unit 102 and/or more than one communication link 103, and/or more than one data processing terminal 105. Multiple sensors may be positioned in a patient for analyte monitoring at the same or different times. In certain embodiments, analyte information obtained by a first positioned sensor may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring system 100 may be a continuous monitoring system, or semi-continuous, or a discrete monitoring system. In a multi-component environment, each component may be configured to be uniquely identified by one or more of the other components in the system so that communication conflict may be readily resolved between the various components within the analyte monitoring system 100. For example, unique IDs, communication channels, and the like, may be used.

In certain embodiments, the sensor 101 is physically positioned in or on the body of a user whose analyte level is being monitored. The sensor 101 may be configured to at least periodically sample the analyte level of the user and convert the sampled analyte level into a corresponding data signal for transmission by the data processing unit 102. The data processing unit 102 is coupleable to the sensor 101 so that both devices are positioned in or on the user's body, with at least a portion of the analyte sensor 101 positioned transcutaneously. The data processing unit 102 may include a fixation element such as adhesive or the like to secure it to the user's body. A mount (not shown) attachable to the user and mateable with the data processing unit 102 may be used. For example, a mount may include an adhesive surface. The data processing unit 102 performs data processing functions, where such functions may include but are not limited to, filtering and encoding of data signals, each of which corresponds to a sampled analyte level of the user, for transmission to the primary receiver unit 104 via the communication link 103. In one embodiment, the sensor 101 or the data processing unit 102 or a combined sensor/data processing unit may be wholly implantable under the skin layer of the user.

In certain embodiments, the analyte monitoring system 100 is configured with a one-way RF communication path from the data processing unit 102 to the primary receiver unit 104. In such embodiments, the data processing unit 102 may transmit the sampled data signals received from the sensor 101 without acknowledgement from the primary receiver unit 104 that the transmitted sampled data signals have been received. For example, the data processing unit 102 may be configured to transmit the encoded sampled data signals at a fixed rate (e.g., at one minute intervals, or other interval) after the completion of an initial power on procedure. Likewise, the primary receiver unit 104 may be configured to detect such transmitted encoded sampled data signals at predetermined time intervals. Alternatively, the analyte monitoring system 100 may be configured with a bi-directional RF (or otherwise) communication between the data processing unit 102 and the primary receiver unit 104.

Additionally, in one aspect, the primary receiver unit 104 may include two sections. The first section is an analog interface section that is configured to communicate with the data processing unit 102 via the communication link 103. In certain embodiments the analog interface section may include an RF receiver and an antenna for receiving and amplifying the data signals from the data processing unit 102, which are thereafter, demodulated with a local oscillator and filtered through a band-pass filter. The second section of the primary receiver unit 104 is a data processing section which is configured to process the data signals received from the data processing unit 102 such as by performing data decoding, error detection and correction, data clock generation, data bit recovery, etc., or any combination thereof.

In operation, upon completing a power-on procedure, the primary receiver unit 104 may be configured to detect the presence of the data processing unit 102 within its range based on predetermined criteria, for example, the strength of the detected data signals received from the data processing unit 102 or predetermined transmitter identification information, or other criteria. Upon successful synchronization with the corresponding data processing unit 102, the primary receiver unit 104 is configured to begin receiving from the data processing unit 102 data signals corresponding to the user's detected analyte level. The primary receiver unit 104, in certain embodiments, is configured to perform synchronized time hopping with the corresponding synchronized data processing unit 102 via the communication link 103 to obtain the user's detected analyte level.

Referring again to FIG. 1, the data processing terminal 105 may include a personal computer, a portable computer such as a laptop or a handheld device (e.g., personal digital assistants (PDAs), telephone such as a cellular phone (e.g., a multimedia and Internet-enabled mobile phone such as an iPhone or similar phone), mp3 player, pager, and the like), or a drug delivery device, each of which may be configured for data communication with the receiver via a wired or a wireless connection. Additionally, the data processing terminal 105 may further be connected to a data network (not shown) for storing and/or retrieving and/or updating and/or analyzing data corresponding to the detected analyte level of the user.

The data processing terminal 105 may include an infusion device such as an insulin infusion pump or the like, which may be configured to administer insulin to patients, and which may be configured to communicate with the receiver unit 104 for receiving, among others, the measured analyte level. Alternatively, the receiver unit 104 may be configured to integrate an infusion device therein so that the receiver unit 104 is configured to administer insulin (or other appropriate drug) therapy to patients, for example, for administering and modifying basal profiles, as well as for determining appropriate boluses for administration based on, among others, the detected analyte levels received from the data processing unit 102. An infusion device may be an external device or an internal device (wholly implantable in a user).

Additionally, the data processing unit 102, the primary receiver unit 104 and the data processing terminal 105 may each be configured for bi-directional wireless communication such that each of the data processing unit 102, the primary receiver unit 104 and the data processing terminal 105 may be configured to communicate (that is, transmit data to and receive data from) with each other via the wireless communication link 103 (or a wired link). More specifically, the data processing terminal 105 may be configured to receive data directly from the data processing unit 102 via a communication link, where the communication link, as described above, may be configured for bi-directional communication.

In such embodiments, the data processing terminal 105, which may include an insulin pump, may be configured to receive the analyte signals from the data processing unit 102, and thus, incorporate the functions of the primary receiver 104 including data processing for managing the patient's insulin therapy and analyte monitoring. In certain embodiments, the communication link 103 as well as one or more of the other communication interfaces shown in FIG. 1, may use one or more of an RF communication protocol, an infrared communication protocol, a Bluetooth enabled communication protocol, an 802.11x wireless communication protocol, or an equivalent wireless communication protocol which would allow secure, wireless communication of several units (for example, per HIPPA requirements) while avoiding potential data collision and interference.

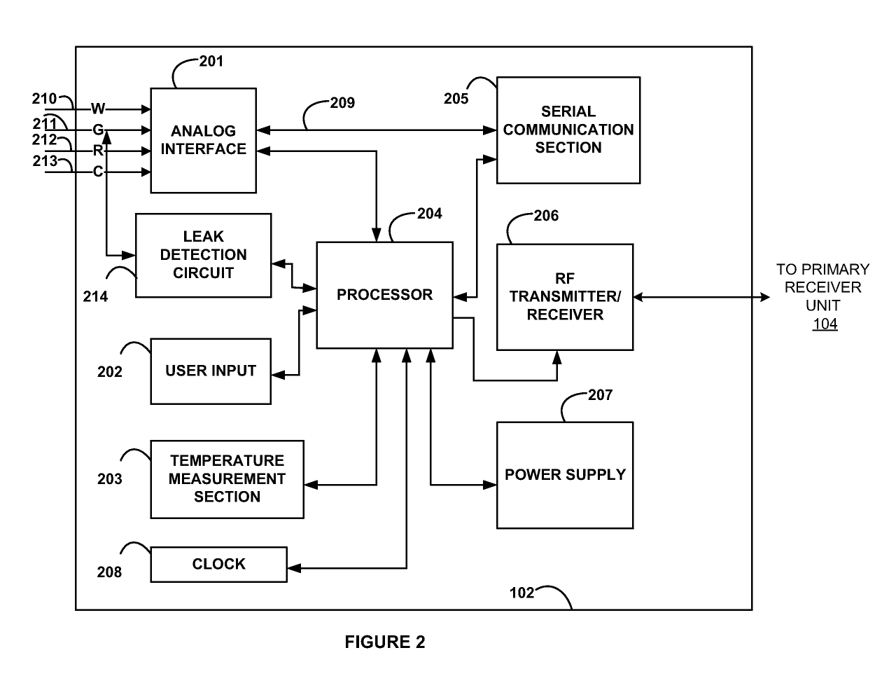

FIG. 2 shows a block diagram of the data processing unit of the data monitoring and detection system shown in FIG. 1 in accordance with certain embodiments. The data processing unit 102 thus may include one or more of an analog interface 201 configured to communicate with the sensor 101 (FIG. 1), a user input 202, and a temperature measurement section 203, each of which is operatively coupled to a processor 204 such as a central processing unit (CPU). The data processing unit 102 may include user input and/or interface components or may be free of user input and/or interface components. In certain embodiments, one or more application-specific integrated circuits (ASIC) may be used to implement one or more functions or routines associated with the operations of the data processing unit (and/or receiver unit) using for example one or more state machines and buffers.

Further shown in FIG. 2 are a transmitter serial communication section 205 and an RF transmitter 206, each of which is also operatively coupled to the processor 204. The RF transmitter 206, in some embodiments, may be configured as an RF receiver or an RF transmitter/receiver, such as a transceiver, to transmit and/or receive data signals. Moreover, a power supply 207, such as a battery, may also be provided in the data processing unit 102 to provide the necessary power for the data processing unit 102. Additionally, as can be seen from the Figure, clock 208 may be provided to, among others, supply real time information to the processor 204.

As can be seen in the embodiment of FIG. 2, the sensor unit 101 (FIG. 1) includes four contacts, three of which are electrodes—work electrode (W) 210, guard contact (G) 211, reference electrode (R) 212, and counter electrode (C) 213, each operatively coupled to the analog interface 201 of the data processing unit 102. In certain embodiments, each of the work electrode (W) 210, guard contact (G) 211, reference electrode (R) 212, and counter electrode (C) 213 may be made using a conductive material that may be applied by, e.g., chemical vapor deposition (CVD), physical vapor deposition, sputtering, reactive sputtering, printing, coating, ablating (e.g., laser ablation), painting, dip coating, etching, and the like. Materials include, but are not limited to, aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

In certain embodiments, a unidirectional input path is established from the sensor 101 (FIG. 1) and/or manufacturing and testing equipment to the analog interface 201 of the data processing unit 102, while a unidirectional output is established from the output of the RF transmitter 206 of the data processing unit 102 for transmission to the primary receiver unit 104. In this manner, a data path is shown in FIG. 2 between the aforementioned unidirectional input and output via a dedicated link 209 from the analog interface 201 to serial communication section 205, thereafter to the processor 204, and then to the RF transmitter 206. As such, in certain embodiments, via the data path described above, the data processing unit 102 is configured to transmit to the primary receiver unit 104 (FIG. 1), via the communication link 103 (FIG. 1), processed and encoded data signals received from the sensor 101 (FIG. 1). Additionally, the unidirectional communication data path between the analog interface 201 and the RF transmitter 206 discussed above allows for the configuration of the data processing unit 102 for operation upon completion of the manufacturing process as well as for direct communication for diagnostic and testing purposes.

The processor 204 may be configured to transmit control signals to the various sections of the data processing unit 102 during the operation of the data processing unit 102. In certain embodiments, the processor 204 also includes memory (not shown) for storing data such as the identification information for the data processing unit 102, as well as the data signals received from the sensor 101. The stored information may be retrieved and processed for transmission to the primary receiver unit 104 under the control of the processor 204. Furthermore, the power supply 207 may include a commercially available battery.

The data processing unit 102 is also configured such that the power supply section 207 is capable of providing power to the data processing unit 102 for a minimum period of time, e.g., at least about one month, e.g., at least about three months or more, of continuous operation. The minimum may be after (i.e., in addition to), a period of time, e.g., up to about eighteen months, of being stored in a low- or no-power (non-operating) mode. In certain embodiments, this may be achieved by the processor 204 operating in low power modes in the non-operating state, for example, drawing no more than minimal current, e.g., approximately 1 μA of current or less. In certain embodiments, a manufacturing process of the data processing unit 102 may place the data processing unit 102 in the lower power, non-operating state (i.e., post-manufacture sleep mode). In this manner, the shelf life of the data processing unit 102 may be significantly improved. Moreover, as shown in FIG. 2, while the power supply unit 207 is shown as coupled to the processor 204, and as such, the processor 204 is configured to provide control of the power supply unit 207, it should be noted that within the scope of the present disclosure, the power supply unit 207 is configured to provide the necessary power to each of the components of the data processing unit 102 shown in FIG. 2.

Referring back to FIG. 2, the power supply section 207 of the data processing unit 102 in one embodiment may include a rechargeable battery unit that may be recharged by a separate power supply recharging unit (for example, provided in the receiver unit 104) so that the data processing unit 102 may be powered for a longer period of usage time. In certain embodiments, the data processing unit 102 may be configured without a battery in the power supply section 207, in which case the data processing unit 102 may be configured to receive power from an external power supply source (for example, a battery, electrical outlet, etc.) as discussed in further detail below.

Referring yet again to FIG. 2, a temperature detection section 203 of the data processing unit 102 is configured to monitor the temperature of the skin near the sensor insertion site. The temperature reading may be used to adjust the analyte readings obtained from the analog interface 201.

The RF transmitter 206 of the data processing unit 102 may be configured for operation in a certain frequency band, e.g., the frequency band of 315 MHz to 322 MHz, for example, in the United States. (The frequency band may be the same or different outside the United States. Further, in certain embodiments, the RF transmitter 206 is configured to modulate the carrier frequency by performing, e.g., Frequency Shift Keying and Manchester encoding, and/or other protocol(s). In certain embodiments, the data transmission rate is set for efficient and effective transmission. For example, in certain embodiments the data transmission rate may be about 19,200 symbols per second, with a minimum transmission range for communication with the primary receiver unit 104.

Also shown is a leak detection circuit 214 coupled to the guard electrode (G) 211 and the processor 204 in the data processing unit 102 of the data monitoring and management system 100. The leak detection circuit 214 may be configured to detect leakage current in the sensor 101 to determine whether the measured sensor data are corrupt or whether the measured data from the sensor 101 is accurate. Such detection may trigger a notification to the user.

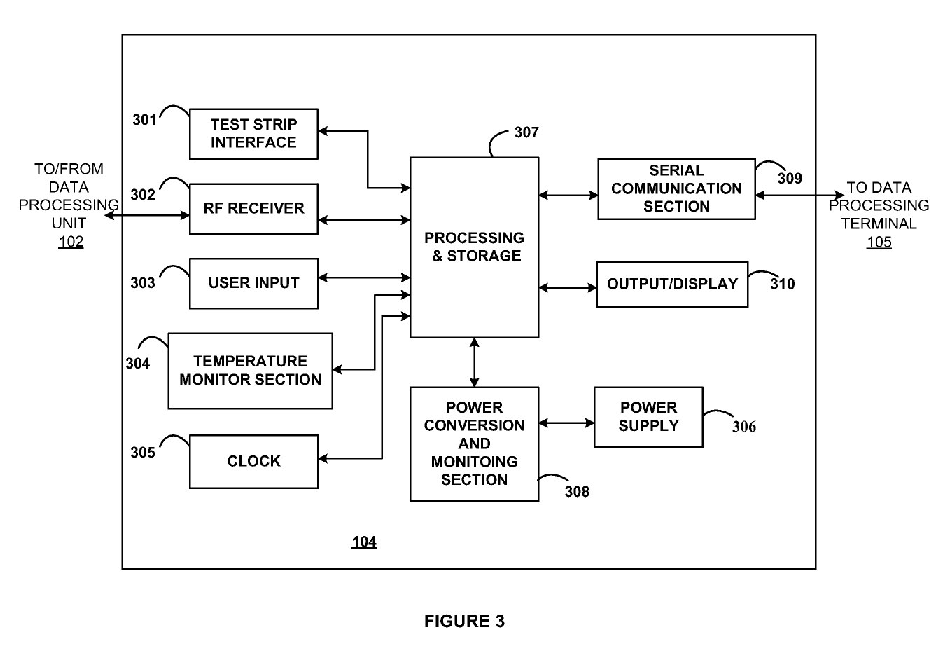

FIG. 3 shows a block diagram of the receiver/monitor unit of the data monitoring and management system shown in FIG. 1 in accordance with certain embodiments. The primary receiver unit 104 includes one or any combination of more than one of: a reference value, e.g., blood glucose test strip, interface 301, an RF receiver 302, an input 303, a temperature monitor section 304, and a clock 305, each of which are operatively coupled to a receiver processing and storage unit 307. The primary receiver unit 104 also includes a power supply 306 operatively coupled to a power conversion and monitoring section 308. Further, the power conversion and monitoring section 308 is also coupled to the receiver processing and storage unit 307. Moreover, also shown are a receiver serial communication section 309, and an output/display 310, each operatively coupled to the receiver processing and storage unit 307. The receiver may include user input and/or interface components or may be free of user input and/or interface components.

In certain embodiments, the reference value, e.g., test strip, interface 301 includes a glucose level testing portion to receive a blood (or other body fluid sample) glucose test or information related thereto. For example, the interface may include a test strip port to receive a glucose test strip. The device may determine the glucose level of the test strip, and optionally display (or otherwise notice) the glucose level on the output 310 of the primary receiver unit 104. Any suitable test strip may be employed, e.g., test strips that only require a very small amount (e.g., one microliter or less, e.g., 0.5 microliter or less, e.g., 0.1 microliter or less), of applied sample to the strip in order to obtain accurate glucose information, e.g. FreeStyle® or Precision® blood glucose test strips from Abbott Diabetes Care Inc. Glucose information obtained by the in vitro glucose testing device may be used for a variety of purposes, computations, etc. For example, the information may be used to calibrate sensor 101, confirm results of the sensor 101 to increase the confidence thereof (e.g., in instances in which information obtained by sensor 101 is employed in therapy related decisions), etc.

The RF receiver 302 is configured to communicate, via the communication link 103 (FIG. 1) with the RF transmitter 206 of the data processing unit 102, to receive encoded data signals from the data processing unit 102 for, among others, signal mixing, demodulation, and other data processing. The input 303 of the primary receiver unit 104 is configured to allow the user to enter information into the primary receiver unit 104 as needed. In one aspect, the input 303 may include keys of a keypad, a touch-sensitive screen, and/or a voice-activated input command unit, and the like. The temperature monitor section 304 is configured to provide temperature information of the primary receiver unit 104 to the receiver processing and storage unit 307, while the clock 305 provides, among others, real time information to the receiver processing and storage unit 307.

Each of the various components of the primary receiver unit 104 shown in FIG. 3 is powered by the power supply 306 (and/or other power supply) which, in certain embodiments, includes a battery. Furthermore, the power conversion and monitoring section 308 is configured to monitor the power usage by the various components in the primary receiver unit 104 for effective power management and may alert the user, for example, in the event of power usage which renders the primary receiver unit 104 in sub-optimal operating conditions. An example of such sub-optimal operating condition may include, for example, operating the vibration output mode (as discussed below) for a period of time thus substantially draining the power supply 306 while the processing and storage unit 307 (thus, the primary receiver unit 104) is turned on. Moreover, the power conversion and monitoring section 308 may additionally be configured to include a reverse polarity protection circuit such as a field effect transistor (FET) configured as a battery activated switch.

The serial communication section 309 in the primary receiver unit 104 is configured to provide a bi-directional communication path from the testing and/or manufacturing equipment for, among others, initialization, testing, and configuration of the primary receiver unit 104. Serial communication section 309 can also be used to upload data to a computer, such as time-stamped blood glucose data. The communication link with an external device (not shown) can be made, for example, by cable, infrared (IR) or RF link. The output 310 of the primary receiver unit 104 is configured to provide, among others, a graphical user interface (GUI) such as a liquid crystal display (LCD) for displaying information. Additionally, the output 310 may also include an integrated speaker for outputting audible signals as well as to provide vibration output as commonly found in handheld electronic devices, such as mobile telephones, pagers, etc. In certain embodiments, the primary receiver unit 104 also includes an electro-luminescent lamp configured to provide backlighting to the output 310 for output visual display in dark ambient surroundings.

Referring back to FIG. 3, the primary receiver unit 104 may also include a storage section such as a programmable, non-volatile memory device as part of the processing and storage unit 307, or provided separately in the primary receiver unit 104, operatively coupled to the processor. The processing and storage unit 307 may be configured to perform Manchester decoding (or other protocol(s)) as well as error detection and correction upon the encoded data signals received from the data processing unit 102 via the communication link 103.

In further embodiments, the data processing unit 102 and/or the primary receiver unit 104 and/or the secondary receiver unit 106, and/or the data processing terminal/infusion section 105 may be configured to receive the blood glucose value wirelessly over a communication link from, for example, a glucose meter. Alternatively, such data or value may be received via a wired or cabled transmission. In further embodiments, a user manipulating or using the analyte monitoring system 100 (FIG. 1) may manually input the blood glucose value using, for example, a user interface (for example, a keyboard, keypad, voice commands, and the like) incorporated in the one or more of the data processing unit 102, the primary receiver unit 104, secondary receiver unit 106, or the data processing terminal/infusion section 105.

In certain embodiments, the data processing unit 102 (FIG. 1) is configured to detect the current signal from the sensor unit 101 (FIG. 1) and optionally the skin and/or ambient temperature near the sensor unit 101, which may be preprocessed by, for example, the data processing unit processor 204 (FIG. 2) and transmitted to the receiver unit (for example, the primary receiver unit 104 (FIG. 1)) at least periodically at a predetermined time interval, such as for example, but not limited to, once per minute, once every two minutes, once every five minutes, or once every ten minutes. Additionally, the data processing unit 102 may be configured to perform sensor insertion detection and data quality analysis, information pertaining to which may also transmitted to the receiver unit 104 periodically at the predetermined time interval. In turn, the receiver unit 104 may be configured to perform, for example, skin temperature compensation as well as calibration of the sensor data received from the data processing unit 102.

Additional detailed descriptions of embodiments of the continuous analyte monitoring system, calibrations protocols, embodiments of its various components are provided in U.S. Pat. Nos. 5,262,035; 5,264,104; 5,262,305; 5,320,715; 5,593,852; 6,175,752; 6,284,478; 6,650,471; 6,746, 582, 7,299,082; application Ser. No. 10/745,878 filed Dec. 26, 2003 entitled “Continuous Glucose Monitoring System and Methods of Use”, the disclosures of each of which are incorporated herein by reference in their entirety.

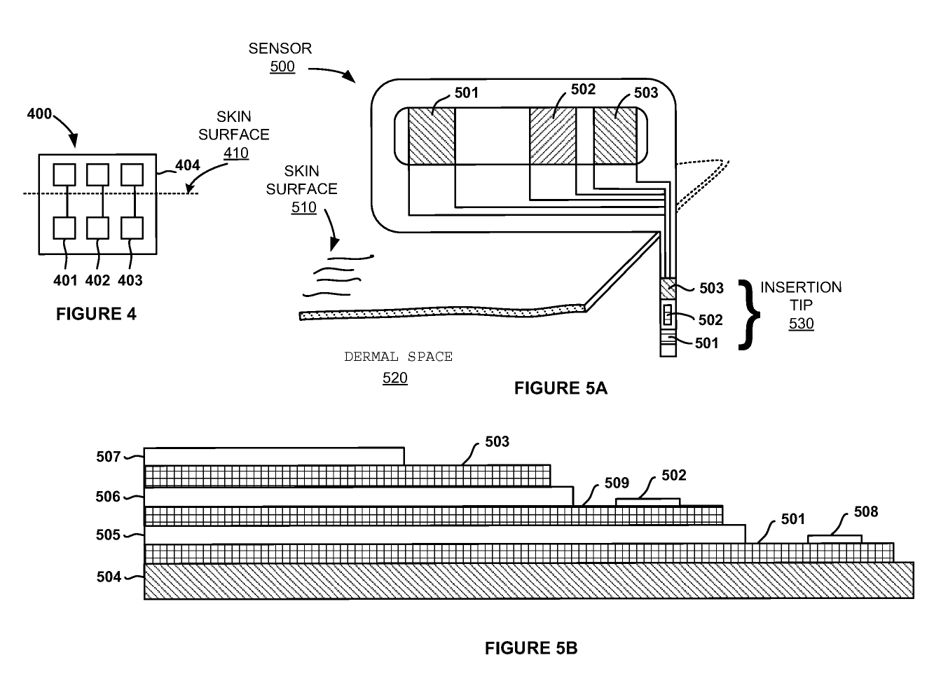

FIG. 4 shows a schematic diagram of an embodiment of an analyte sensor in accordance with the present disclosure. The sensor 400 includes electrodes 401, 402 and 403 on a base 404. It is to be understood that the sensor may be configured as a wire, e.g., as described in, e.g., U.S. Pat. No. 6,284,478. The sensor may be configured to be wholly implantable in a user or may be configured so that only a portion is positioned within (internal) a user and another portion outside (external) a user. For example, the sensor 400 may include a portion positionable above a surface of the skin 410, and a portion positioned below the skin (i.e., positioned transcutaneously), for example, in the dermal layer. In such embodiments, the external portion may include contacts (connected to respective electrodes of the second portion, e.g., by traces) to connect to another device also external to the user such as a sensor control unit, e.g., a transmitter unit. While the embodiment of FIG. 4 shows three electrodes side-by-side on the same surface of base 404, other configurations are contemplated, e.g., fewer or greater electrodes, some or all electrodes on different surfaces of the base or present on another base, some or all electrodes stacked together, electrodes of differing materials and dimensions, etc.

FIG. 5A shows a perspective view of an embodiment of an electrochemical analyte sensor 500 having a first portion (which in this embodiment may be characterized as a major portion) positionable above a surface of the skin 510, and a second portion (which in this embodiment may be characterized as a minor portion) that includes an insertion tip 530 positionable below the skin, e.g., penetrating through the skin and into, e.g., the dermal space 520, in contact with the user's biofluid such as dermal fluid. While in this particular embodiment the sensor is shown having a “minor” poritions and a “major” portion, embodiments include sensors that have portions that are substantially the same, e.g., in one or more, including all, dimensions. In one aspect, the second portion 520 may include a length that does not exceed approximately 3 millimeters in length such that upon transcutaneous insertion of the analyte sensor 500, the portion that resides under the skin surface, e.g., tip 530, of the analyte sensor 500 does not enter the subcutaneous layer under the skin. In this manner, the insertion of the analyte sensor 500 results in positioning of at least the working electrode 501 within the dermal fluid under the skin surface.

Contact portions of a working electrode 501, a reference electrode 502, and a counter electrode 503 are positioned on the portion of the sensor 500 situated above the skin surface 510. In this embodiment, working electrode 501, a reference electrode 502, and a counter electrode 503 are shown at the second section and particularly at the insertion tip 530. Traces, if necessary, may be provided from the electrode at the tip to the contact, as shown in FIG. 5A. It is to be understood that greater or fewer electrodes may be provided on a sensor. For example, a sensor may include more than one working electrode and/or the counter and reference electrodes may be a single counter/reference electrode, etc.

FIG. 5B shows a cross sectional view of a portion of the sensor 500 of FIG. 5A. The electrodes 501, 502 and 503, of the sensor 500 as well as the substrate and the dielectric layers are provided in a layered configuration or construction. For example, as shown in FIG. 5B, in one aspect, the sensor 500 (such as the sensor unit 101 FIG. 1), includes a substrate layer 504, and a first conducting layer 501 such as carbon, gold, etc., disposed on at least a portion of the substrate layer 504, and which may provide the working electrode. Also shown disposed on at least a portion of the first conducting layer 501 is a sensing layer 508.

Referring back to FIG. 5B, a first insulation layer such as a first dielectric layer 505 is disposed or layered on at least a portion of the first conducting layer 501, and further, a second conducting layer 509 may be disposed or stacked on top of at least a portion of the first insulation layer (or dielectric layer) 505. As shown in FIG. 5B, the second conducting layer 509 may provide the reference electrode 502, and in one aspect, may include a layer of silver/silver chloride (Ag/AgCl), gold, etc.

Referring still again to FIG. 5B, a second insulation layer 506 such as a dielectric layer in one embodiment may be disposed or layered on at least a portion of the second conducting layer 509. Further, a third conducting layer 503 may provide the counter electrode 503. It may be disposed on at least a portion of the second insulation layer 506. Finally, a third insulation layer 507 may be disposed or layered on at least a portion of the third conducting layer 503. In this manner, the sensor 500 may be layered such that at least a portion of each of the conducting layers is separated by a respective insulation layer (for example, a dielectric layer).

The embodiment of FIGS. 5A and 5B show the layers having different lengths. Some or all of the layers may have the same or different lengths and/or widths.

In certain embodiments including where the sensor is inserted at an angle approximately greater than 30 degrees relative to the skin surface, the working electrode on the substrate 504 may be provided such that it is located closer to the skin surface as compared with the location of the counter and/or reference electrodes 503, 502. That is, in certain embodiments, the working electrode with sensing layer disposed thereon may be provided on the sensor substrate such that transcutaneous positioning of the sensor will ensure that the working electrode is positioned within the dermal layer under the skin surface.

In certain embodiments, some or all of the electrodes 501, 502, 503 may be provided on the same side of the substrate 504 in the layered construction as described above, or alternatively, may be provided in a co-planar manner such that two or more electrodes may be positioned on the same plane (e.g., side-by side (e.g., parallel) or angled relative to each other) on the substrate 504. For example, co-planar electrodes may include a suitable spacing there between and/or include dielectric material or insulation material disposed between the conducting layers/electrodes. Furthermore, in certain embodiments one or more of the electrodes 501, 502, 503 may be disposed on opposing sides of the substrate 504. In such embodiments, contact pads may be on the same or different sides of the substrate. For example, an electrode may be on a first side and its respective contact may be on a second side, e.g., a trace connecting the electrode and the contact may traverse through the substrate.

Within the scope of the present disclosure, the analyte sensor may be fabricated using MEMS technology such that the sensors includes microneedles or microprojections, where a microprojection configuration may include a solid microneedle-like structure without the central bore through which liquid is injected or withdrawn in a microneedle. Sensors may be fabricated using this or similar structures, such that the sensors include microneedles or microprojections having electrodes and/or sensing chemistry disposed at the penetrating tip of each microneedle or microprojection, where the microneedles are constructed such that when placed on the skin surface, the electrodes on the tips of the microneedles or microprojections are positioned within the dermal layer under the skin surface, for example, at a depth not exceeding 1 millimeter, and for example, approximately 300 micrometers.

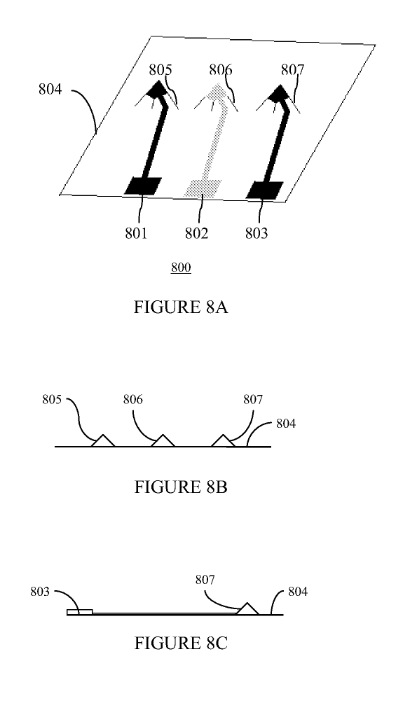

FIGS. 8A-8C schematically show a perspective view and side cross sectional views of an embodiment of an analyte sensor with microprojection configuration in accordance with one aspect of the present disclosure. The sensor 800 in certain embodiments may include contact pads 801, 802 and 803 on a base 804. The contact pads 801, 802, 803 are connected via conducting traces to electrodes 805, 806, and 807 (working, reference and counter electrodes respectively in this embodiment) which are situated atop the microprotrusions.

In certain aspects, external connection of measuring electronics (for example, the data processing unit 102 (FIG. 1)) may be provided to the contact pads 801, 802, 803. The working electrode 805 in one aspect may be provided with an analyte sensing layer. The reference electrode 806 may be constructed of Ag/AgCl or other suitable material. When the sensor 800 is placed against the skin surface, and pressure is applied, the electrode-carrying microprotrusions penetrate through the outer surface of the skin layer and until they reach the dermal layer, where they sense or monitor the presence of analyte in the dermal fluid. While the embodiment of FIG. 8 shows three electrodes side-by-side on the same surface of base 804, other configurations are contemplated, e.g., fewer or greater electrodes, some or all electrodes on different surfaces of the base or present on another base, some or all electrodes stacked together, electrodes of differing materials and dimensions, etc. In one aspect, the working electrode is configured or positioned to reside within the dermal layer, while other electrodes (counter, references, or counter/reference) may be positioned to reside in the deeper subcutaneous layer, on the skin surface, or in some other part of the body.

In particular, a microneedle array sensor configuration may include a doped silicon substrate with silicon oxide, or other insulating layer disposed thereon to insulate the surfaces and the sides of the microneedles, with an orifice at the tip of the microneedle to provide electrical connection to the doped silicon. Thereafter, a layer of gold, platinium-iridium or other suitable material may be disposed on each microneedle to form a conductive layer over the microneedle and the immediate base section while exposing the tip of each microneedle. The microneedle tip may be provided with one or more sensing layers to form, for example, the working electrode and sensing chemistry disposed thereon.

In another embodiment, the microprojections or microneedles may be fabricated from an insulating material, with individually addressable conducting electrodes mounted atop each skin penetrating microprojection or microneedle, as in FIGS. 8A-8C. Such a configuration may be equipped with additional insulating layers over the electrode traces to ensure uniform electrode areas.

In a further embodiment, the microneedle sensor array configuration may include channels disposed thereon to direct fluid such as medication including, for example, insulin or other therapeutic agent that may be used in conjunction with the analyte monitoring system.

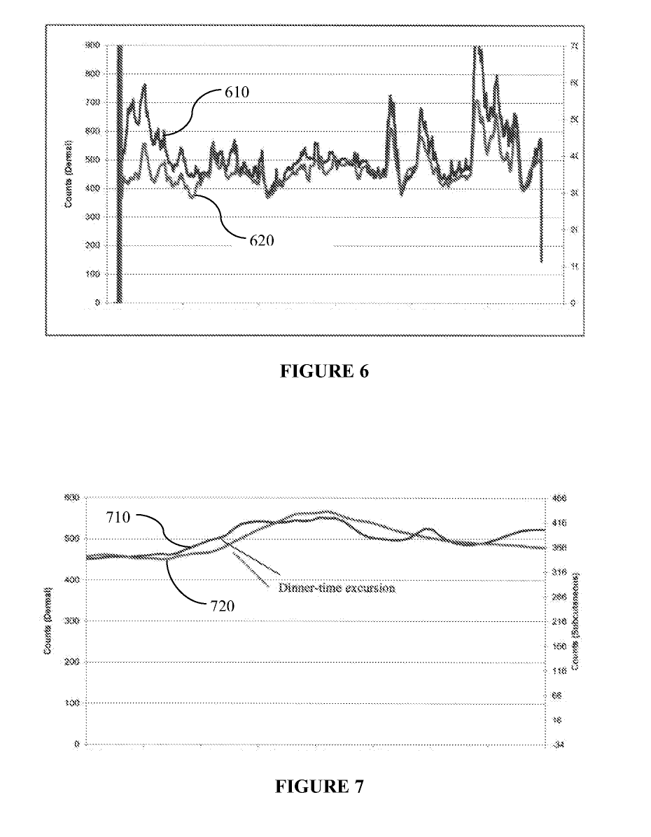

FIG. 6 is a graphical illustration of the monitored analyte level with a sensor positioned in the dermal layer compared to a sensor positioned in the interstitial fluid. Referring to FIG. 6, the results of monitored analyte levels using two sensors including a first glucose sensor with subcutaneously positioned working electrode (hereinafter referred to as “subcutaneous sensor”), and a second sensor with working electrode that is positioned in the dermal fluid (“hereinafter referred to as “dermal sensor”), in the same subject, is shown over a period of approximately 36 hours. The subcutaneous sensor's working electrode was positioned at approximately 5 millimeters below the skin surface, while the dermal sensor's working electrode was positioned at approximately 1 millimeter below the skin surface (substantially parallel to the skin layer).

Referring to FIG. 6, there are illustrated two Y-axis scales showing raw current signal level illustrates as signal counts, with the left Y-axis showing the signal count for the dermal sensor scale while that in the right Y-axis shows the signal count for the subcutaneous sensor, where the two scales are normalized for sensitivity differences (and X-axis being time). Referring to the Figure, it can be seen that the subcutaneous sensor 620 illustrates reduced sensitivity during the first six to eight hours of sensor positioning, while the dermal sensor 610 illustrated a relatively wider dynamic range.

Indeed, as can be seen from FIG. 6, the response time for monitoring or detecting analyte level from the dermal sensor is relatively faster (for example, during the first 6-8 hours from sensor insertion), as compared to the response time for monitoring or detecting analyte level from the subcutaneous sensor from the same user/person. In other words, in the same subject or user, the dermal sensor yielded signals corresponding to the monitored or detected analyte level relatively faster or sooner than the subcutaneous sensor measured from the same sensor insertion time period, such that a faster physiological response time was attained from the dermal sensor as compared to the subcutaneous sensor.

FIG. 7 is a graphical illustration of dinner time rise in the analyte sensor response based on the dermal sensor in comparison to the subcutaneous sensor. Referring to FIG. 7, it can be seen that the dermal sensor 710 responded faster than the subcutaneous sensor 720 as shown in FIG. 7 by the region labeled “dinner time excursion”.

Indeed, as shown above, the dermal sensor has a faster physiological response time as compared to the subcutaneous sensor.

In aspects of the present disclosure, shallow placement of an analyte sensor for positioning the dermal layer under the skin surface of a user is provided. For example, analyte sensors positioned in the dermal layer (typically approximately 2 mm under the skin surface) in embodiments of the present disclosure results in minimal insertion trauma based on reduced insertion depth, and also results in a more rapid response as compared to an analyte sensor positioned in the subcutaneous tissue.

Aspects include positioning the working electrode of the analyte sensor in the dermal layer, for example, at a predetermined angle relative to the skin surface, including substantially parallel, substantially 45°, substantially 90°, relative to the skin surface. Reference and/or counter electrodes of the analyte sensor may likewise be positioned in the dermal layer.

Embodiments include analyte monitoring devices and systems that include an analyte sensor—at least a portion of which is positionable beneath the skin of the user—for the in vivo detection, of an analyte, such as glucose, lactate, and the like, in a body fluid. Embodiments include wholly implantable analyte sensors and analyte sensors in which only a portion of the sensor is positioned under the skin and a portion of the sensor resides above the skin, e.g., for contact to a transmitter, receiver, transceiver, processor, etc. The sensor may be, for example, positionable in a patient for the continuous or periodic monitoring of a level of an analyte in a patient's dermal fluid.

In certain embodiments, the working electrode of an analyte sensor may be positioned in contact with dermal fluid to detect the level of glucose, which detected glucose may be used to infer the glucose level in the patient's bloodstream. Analyte sensors may be insertable through the skin layer and into the dermal layer under the skin surface at a depth of approximately 3 mm under the skin surface and containing dermal fluid. Embodiments of the analyte sensors of the subject disclosure may be configured for monitoring the level of the analyte over a time period which may range from minutes, hours, days, weeks, or longer.

Accordingly, a method in one aspect includes transcutaneously positioning an analyte sensor through a skin layer of a user, said analyte sensor including an implanted portion having a predetermined length, where said implanted portion of the analyte sensor is positioned within a dermal layer under the skin layer, and detecting a signal generated from the implanted portion of the analyte sensor, wherein the signal is associated with an analyte level of the user.

In one aspect, transcutaneously positioning the analyte sensor may include positioning the implanted portion at a depth under the skin layer not exceeding approximately 3 millimeters.

The implanted portion of the analyte sensor may be positioned within the dermal layer substantially in parallel to the skin layer.

The implanted portion of the analyte sensor may be substantially in fluid contact with dermal fluid of the user.

The analyte level may include glucose level.

Further, transcutaneous positioning the analyte sensor may include manually penetrating the skin layer.

The implanted portion of the analyte sensor may include one or more electrodes in fluid contact with dermal fluid of the user, where the one or more electrodes may include a working electrode.

The implanted portion of the analyte sensor may be positioned under the skin layer at a depth not exceeding approximately 2 millimeters.

In still another aspect, the implanted portion of the analyte sensor may be positioned under the skin layer at a depth not exceeding approximately 1 millimeter.

An analyte sensor insertion device in another aspect includes an introducer having a sharp end, and an analyte sensor having a predetermined length and coupled to the introducer for positioning, e.g., wholly or transcutaneously, of at least a portion of the analyte sensor under a skin layer of a user at a predetermined depth so that at least the working electrode is positioned in the dermal layer, e.g., the insertion device is configured to deploy the sensor so that it does not exceed (or at least the position of the working electrode does not exceed) approximately 3 millimeters under the skin, where the portion of the analyte sensor under the skin layer includes one or more electrodes in fluid contact with dermal fluid for detecting a signal from the dermal fluid associated with an analyte level of the user. For example, the inserter may include a depth control mechanism coupled to the sharp that accomplishes the above so that the portion of the sensor where the analyte chemical reaction occurs is deployed substantially at the dermal layer depth and not beyond.

In one aspect, the sharp end may be beveled.

In another aspect, the sharp end may include a polished surface.

The introducer may be configured to manually pierce the skin layer.

The introducer may be removed from the user after positioning the analyte sensor.

The introducer may include a substantially hollow needle, and further, where at least a portion of the analyte sensor is disposed within the hollow needle during transcutaneous positioning.

The introducer may be configured to position the portion of analyte sensor at the predetermined depth at a predefined angle relative to the skin layer, where the predefined angle may be less than or equal to about 90 degrees, for example, but not limited to, 75 degrees, 45 degrees, 15-25 degrees, or less than 5-10 degrees.

In one aspect, the positioned sensor may be retained after transcutaneously positioned under the skin layer such that at least the working electrode of the sensor is retained within the dermal layer. In certain embodiments, a mounting unit positioned on the skin surface may be configured to retain the positioned sensor in place so that the working electrode is substantially held in place in the dermal layer after transcutaneous positioning. Example embodiments of mounting unit is described in further detail in U.S. Pat. No. 6,175,752 and elsewhere, disclosure of which is incorporated herein by reference. Alternatively or in addition, a layer of adhesive may be applied or provided over the positioned sensor so as to retain the sensor in the desired location, for example, such that the working electrode is held in place in the dermal layer under the skin.

The introducer may include a microneedle assembly.

The analyte may be is disposed on the sharp end of the microneedle assembly.

A method in still another aspect may include positioning a portion of a transcutaneous analyte sensor under a skin layer of a user to retain one or more electrodes of the analyte sensor intradermally under the skin layer, and receiving one or more analyte associated signals from the one or more electrodes of the analyte sensor.

The portion of the transcutaneous analyte sensor positioned under the skin layer may be at a predefined angle relative to the skin layer, where the predefined angle may include less than approximately 90 degrees, or less than approximately 180 degrees.

The one or more electrodes retained intradermally under the skin layer may include a working electrode.

The portion of the transcutaneous analyte sensor may be positioned under the skin layer at a depth not exceeding approximately 3 millimeters from the skin layer.

The method in another aspect may include securing the positioned analyte sensor such that the one or more electrodes of the analyte sensor is intradermally retained under the skin layer for a predetermined time period, where the predetermined time period may include approximately one day or more, approximately three days or more, approximately seven days or more, approximately one month or more.

The method in still another aspect may include transmitting the received one or more analyte associated signals to a remote location, and where the received one or more analyte associated signals may be wirelessly transmitted.

A kit in accordance with still yet another embodiment includes an insertion device including: a housing, an insertion mechanism coupled to the housing, and an introducer coupled to the insertion mechanism, and an analyte sensor coupled to the introducer, the analyte sensor including an implantable portion with one or more electrodes for intradermal positioning under a skin layer of a user, where upon actuation of the insertion mechanism, the introducer is configured pierce the skin layer of the user to transcutaneously position the implantable portion of the analyte sensor such that at least one of the one or more electrodes is in fluid contact with the dermal fluid of the user, and further, where the introducer is removed from the user after positioning the implantable portion of the analyte sensor.

The insertion mechanism may be spring biased.

The insertion mechanism may be configured to automatically retract the introducer from the user after positioning the implantable portion of the analyte sensor in the dermal layer of the user.

The insertion mechanism may be configured to position the analyte sensor at a predetermined angle relative to the skin layer, where the predetermined angle may be less than or equal to approximately 180 degrees.

The analyte sensor may be positioned substantially parallel to the skin layer.

The portion of the analyte sensor may be transcutaneously positioned under skin layer such that a penetration depth of the introducer and the sensor is maintained above the subcutaneous layer of the user.

The one or more electrodes may include a working electrode.

The analyte sensor may be configured to generate one or more signals associated with an analyte level of the user from the one or more electrodes in fluid contact with the dermal fluid.

The analyte sensor may include one or more of a glucose sensor or an oxygen sensor.

As noted above, analyte sensors include an analyte-responsive enzyme to provide a sensing component or sensing layer. Some analytes, such as oxygen, can be directly electrooxidized or electroreduced on a sensor, and more specifically at least on a working electrode of a sensor. Other analytes, such as glucose and lactate, require the presence of at least one electron transfer agent and/or at least one catalyst to facilitate the electrooxidation or electroreduction of the analyte. Catalysts may also be used for those analyte, such as oxygen, that can be directly electrooxidized or electroreduced on the working electrode. For these analytes, each working electrode includes a sensing layer (see for example sensing layer 408 of FIG. 5B) formed proximate to or on a surface of a working electrode. In many embodiments, a sensing layer is formed near or on only a small portion of at least a working electrode.

The sensing layer includes one or more components designed to facilitate the electrolysis of the analyte. The sensing layer may include, for example, a catalyst to catalyze a reaction of the analyte and produce a response at the working electrode, an electron transfer agent to indirectly or directly transfer electrons between the analyte and the working electrode, or both.

A variety of different sensing layer configurations may be used. In certain embodiments, the sensing layer is deposited on the conductive material of a working electrode. The sensing layer may extend beyond the conductive material of the working electrode. In some cases, the sensing layer may also extend over other electrodes, e.g., over the counter electrode and/or reference electrode (or counter/reference is provided). The sensing layer may be integral with the material of an electrode.

For example, a glucose or lactate sensor may include a first sensing layer which is spaced apart from the working electrode and contains an enzyme, for example, glucose oxidase or lactate oxidase. The reaction of glucose or lactate in the presence of the appropriate enzyme forms hydrogen peroxide. A second sensing layer may be provided directly on the working electrode and contains a peroxidase enzyme and an electron transfer agent to generate a signal at the electrode in response to the hydrogen peroxide. The level of hydrogen peroxide indicated by the sensor then correlates to the level of glucose or lactate.

Another sensor which operates similarly can be made using a single sensing layer with both the glucose or lactate oxidase and the peroxidase being deposited in the single sensing layer. Examples of such sensors are described in U.S. Pat. No. 5,593,852, U.S. patent application Ser. No. 08/540,789, and PCT Patent Application No. US98/02403 entitled “Soybean Peroxidase Electrochemical Sensor”, filed on Feb. 11, 1998, incorporated herein by reference.

A sensing layer that is in direct contact with the working electrode may contain an electron transfer agent to transfer electrons directly or indirectly between the analyte and the working electrode, and/or a catalyst to facilitate a reaction of the analyte. For example, a glucose, lactate, or oxygen electrode may be formed having a sensing layer which contains a catalyst, such as glucose oxidase, lactate oxidase, or laccase, respectively, and an electron transfer agent that facilitates the electrooxidation of the glucose, lactate, or oxygen, respectively.

In other embodiments the sensing layer is not deposited directly on the working electrode. Instead, the sensing layer 64 may be spaced apart from the working electrode, and separated from the working electrode, e.g., by a separation layer. A separation layer may include one or more membranes or films or a physical distance. In addition to separating the working electrode from the sensing layer the separation layer may also act as a mass transport limiting layer and/or an interferent eliminating layer and/or a biocompatible layer.

In certain embodiments which include more than one working electrode, one or more of the working electrodes do not have a corresponding sensing layer, or have a sensing layer which does not contain one or more components (e.g., an electron transfer agent and/or catalyst) needed to electrolyze the analyte. Thus, the signal at this working electrode corresponds to background signal which may be removed from the analyte signal obtained from one or more other working electrodes that are associated with fully-functional sensing layers by, for example, subtracting the signal.

In certain embodiments, the sensing layer includes one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Some quinones and partially oxidized quinhydrones react with functional groups of proteins such as the thiol groups of cysteine, the amine groups of lysine and arginine, and the phenolic groups of tyrosine which may render those redox species unsuitable for some of the sensors of the present disclosure because of the presence of the interfering proteins in an analyte-containing fluid. In many instances substituted quinones and molecules with quinoid structure are less reactive with proteins may be employed. A tetrasubstituted quinone usually has carbon atoms in positions 1, 2, 3, and 4. Examples of organometallic redox species are metallocenes such as ferrocene. Examples of inorganic redox species are hexacyanoferrate (III), ruthenium hexamine etc.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic, or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

One type of polymeric electron transfer agent contains a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation. Another example of an ionically-bound mediator is a positively charged polymer such as quaternized poly(4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, such as 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole).

Embodiments may employ electron transfer agents having a redox potential ranging from about −200 mV to about +200 mV versus the standard calomel electrode (SCE). The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, such as a glucose oxidase, glucose dehydrogenase (e.g., pyrroloquinoline quinone (PQQ) dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent (which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

Certain embodiments include a Wired Enzyme™ sensing layer that works at a gentle oxidizing potential, e.g., a potential of about +40 mV. This sensing layer uses an osmium (Os)-based mediator designed for low potential operation and is stably anchored in a polymeric layer. Accordingly, in certain embodiments the sensing element is redox active component that includes (1) Osmium-based mediator molecules attached by stable (bidente) ligands anchored to a polymeric backbone, and (2) glucose oxidase enzyme molecules. These two constituents are crosslinked together.

A mass transport limiting layer (not shown), e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may serve many functions, e.g., functionalities of a biocompatible layer and/or interferent-eliminating layer may be provided by the mass transport limiting layer.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

According certain embodiments, a membrane is formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly(ethylene glycol), hydroxyl or polyhydroxyl modifiers, may be used to enhance the biocompatibility of the polymer or the resulting membrane.

A membrane may be formed in situ by applying an alcohol-buffer solution of a crosslinker and a modified polymer over an enzyme-containing sensing layer and allowing the solution to cure for one to two days. The crosslinker-polymer solution may be applied to the sensing layer by placing a droplet or droplets of the solution on the sensor, by dipping the sensor into the solution, or the like. Generally, the thickness of the membrane is controlled by the concentration of the solution, by the number of droplets of the solution applied, by the number of times the sensor is dipped in the solution, or by any combination of these factors. A membrane applied in this manner may have any combination of the following functions: (1) mass transport limitation, i.e., reduction of the flux of analyte that can reach the sensing layer, (2) biocompatibility enhancement, or (3) interferent reduction.

The electrochemical sensors may employ any suitable measurement technique. For example, may detect current or may employ potentiometry. Technique may include, but are not limited to amperometry, coulometry, voltammetry. In some embodiments, sensing systems may be optical, calorimetric, and the like.

In certain embodiments, the sensing system detects hydrogen peroxide to infer glucose levels. For example, a hydrogen peroxide-detecting sensor may be constructed in which a sensing layer includes enzyme such as glucose oxides, glucose dehydrogensae, or the like, and is positioned proximate to the working electrode. The sending layer may be covered by a membrane that is selectively permeable to glucose. Once the glucose passes through the membrane, it is oxidized by the enzyme and reduced glucose oxidase can then be oxidized by reacting with molecular oxygen to produce hydrogen peroxide.

Certain embodiments include a hydrogen peroxide-detecting sensor constructed from a sensing layer prepared by crosslinking two components together, for example: (1) a redox compound such as a redox polymer containing pendent Os polypyridyl complexes with oxidation potentials of about +200 mV vs. SCE, and (2) periodate oxidized horseradish peroxidase (HRP). Such a sensor functions in a reductive mode; the working electrode is controlled at a potential negative to that of the Os complex, resulting in mediated reduction of hydrogen peroxide through the HRP catalyst.

In another example, a potentiometric sensor can be constructed as follows. A glucose-sensing layer is constructed by crosslinking together (1) a redox polymer containing pendent Os polypyridyl complexes with oxidation potentials from about −200 mV to +200 mV vs. SCE, and (2) glucose oxidase. This sensor can then be used in a potentiometric mode, by exposing the sensor to a glucose containing solution, under conditions of zero current flow, and allowing the ratio of reduced/oxidized Os to reach an equilibrium value. The reduced/oxidized Os ratio varies in a reproducible way with the glucose concentration, and will cause the electrode's potential to vary in a similar way.

A sensor may also include an active agent such as an anticlotting and/or antiglycolytic agent(s) disposed on at least a portion a sensor that is positioned in a user. An anticlotting agent may reduce or eliminate the clotting of blood or other body fluid around the sensor, particularly after insertion of the sensor. Blood clots may foul the sensor or irreproducibly reduce the amount of analyte which diffuses into the sensor. Examples of useful anticlotting agents include heparin and tissue plasminogen activator (TPA), as well as other known anticlotting agents. Embodiments may include an antiglycolytic agent or precursor thereof. Examples of antiglycolytic agents are glyceraldehyde, fluoride ion, and mannose. The term “antiglycolytic” is used broadly herein to include any substance that at least retards glucose consumption of living cells.

Sensors described herein may be configured to require no calibration or no user calibration. For example, a sensor may be factory calibrated and need not require further calibrating. In certain embodiments, calibration may be required, but may be done without user intervention, i.e., may be automatic. In those embodiments in which calibration by the user is required, the calibration may be according to a predetermined schedule or may be dynamic, i.e., the time for which may be determined by the system on a real-time basis according to various factors, such as but not limited to glucose concentration and/or temperature and/or rate of change of glucose, etc.

Calibration may be accomplished using an in vitro test strip or other calibrator, e.g., a small sample test strip such as a test strip that requires less than about 1 microliter of sample (for example FreeStyle blood glucose monitoring test strips from Abbott Diabetes Care Inc.). For example, test strips that require less than about 1 nanoliter of sample may be used. As noted above, a glucose meter to read a test strip may be coupled to or integrated with a component of a system, e.g., a receiver unit may include a test strip receiving opening in connection with meter componentry.

In certain embodiments, a sensor may be calibrated using only one sample of body fluid per calibration event. For example, a user using need only lance a body part one time to obtain sample for a calibration event (e.g., for a test strip), or may lance more than one time within a short period of time if an insufficient volume of sample is obtained firstly. Embodiments include obtaining and using multiple samples of body fluid for a given calibration event, where glucose values of each sample are substantially similar. Data obtained from a given calibration event may be used independently to calibrate or combined with data obtained from previous calibration events, e.g., averaged including weighted averaged, etc., to calibrate.

An analyte system may include an optional alarm system that, e.g., based on information from a processor, warns the patient of a potentially detrimental condition of the analyte. For example, if glucose is the analyte, an alarm system may warn a user of conditions such as hypoglycemia and/or hyperglycemia and/or impending hypoglycemia, and/or impending hyperglycemia. An alarm system may be triggered when analyte levels reach or exceed a threshold value. An alarm system may also, or alternatively, be activated when the rate of change or acceleration of the rate of change, in analyte level increase or decrease approaches, reaches or exceeds a threshold rate or acceleration. For example, in the case of a glucose monitoring system, an alarm system may be activated if the rate of change in glucose concentration exceeds a threshold value which might indicate that a hyperglycemic or hypoglycemic condition is likely to occur. A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

The subject disclosure also includes sensors used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a processing unit including, in one embodiment, a transmitter unit, a receiver/display unit, and a drug administration system. In some cases, some or all components may be integrated in a single unit. The sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or implanted insulin pump.

What is claimed is:

1. A method, comprising:
transcutaneously positioning an analyte sensor through a skin layer of a user, said analyte sensor including dermal a portion configured to be positioned substantially within a dermal layer under the skin layer; and
detecting a signal generated from the implanted portion of the analyte sensor, wherein the signal is associated with an analyte level of the user.

2. The method of claim 1 wherein transcutaneously positioning the analyte sensor includes positioning the implanted portion at a depth under the skin layer not exceeding approximately 3 millimeters.

3. The method of claim 1 wherein the implanted portion of the analyte sensor is positioned within the dermal layer substantially in parallel to the skin layer.

4. The method of claim 1 wherein the implanted portion of the analyte sensor is substantially in fluid contact with dermal fluid of the user.

5. The method of claim 1 wherein the analyte level includes glucose level.

6. The method of claim 1 wherein transcutaneous positioning the analyte sensor includes manually penetrating the skin layer.

7. The method of claim 1 wherein the implanted portion of the analyte sensor includes one or more electrodes in fluid contact with dermal fluid of the user.

8. The method of claim 7 wherein one or more electrodes includes a working electrode.

9. The method of claim 1 wherein the implanted portion of the analyte sensor is positioned under the skin layer at a depth not exceeding approximately 2 millimeters.

10. The method of claim 1 wherein the implanted portion of the analyte sensor is positioned under the skin layer at a depth not exceeding approximately 1 millimeter.

11. An analyte sensor insertion device, comprising:
an introducer having a sharp end; and
an analyte sensor having a predetermined length and coupled to the introducer for transcutaneous positioning of a portion of the analyte sensor under a skin layer of a user at a predetermined depth not exceeding approximately 3 millimeters;
wherein the portion of the analyte sensor under the skin layer includes one or more electrodes in fluid contact with dermal fluid for detecting a signal from the dermal fluid associated with an analyte level of the user.

12. The device of claim 11 wherein the sharp end is beveled.

13. The device of claim 11 wherein the sharp end has a polished surface.

14. The device of claim 11 wherein the introducer is configured to manually pierce the skin layer.

15. The device of claim 11 wherein the introducer is removed from the user after positioning the analyte sensor.

16. The device of claim 11 wherein the introducer includes a substantially hollow needle, and further, wherein at least a portion of the analyte sensor is disposed within the hollow needle during transcutaneous positioning.

17. The device of claim 11 wherein the introducer is configured to position the portion of analyte sensor at the predetermined depth at a predefined angle relative to the skin layer.

18. The device of claim 17 wherein the predefined angle is less than or equal to 90 degrees.

19. The device of claim 11 wherein the introducer includes a microneedle assembly.

20. The device of claim 11 wherein the analyte sensor is disposed on the sharp end of the microneedle assembly.

21. A method, comprising:
positioning a portion of a transcutaneous analyte sensor under a skin layer of a user to retain one or more electrodes of the analyte sensor intradermally under the skin layer; and
receiving one or more analyte associated signals from the one or more electrodes of the analyte sensor.

22. The method of claim 21 wherein the portion of the transcutaneous analyte sensor positioned under the skin layer is at a predefined angle relative to the skin layer.

23. The method of claim 22 wherein the predefined angle includes less than approximately 90 degrees.

24. The method of claim 22 wherein the predefined angle includes less than approximately 180 degrees.

25. The method of claim 21 wherein the one or more electrodes retained intradermally under the skin layer includes a working electrode.

26. The method of claim 21 wherein the portion of the transcutaneous analyte sensor is positioned under the skin layer at a depth not exceeding approximately 3 millimeters from the skin layer.

27. The method of claim 21 including securing the positioned analyte sensor such that the one or more electrodes of the analyte sensor is intradermally retained under the skin layer for a predetermined time period.

28. The method of claim 27 wherein the predetermined time period includes one of approximately one day, three days or more, or seven days or less.

29. The method of claim 21 including transmitting the received one or more analyte associated signals to a remote location.

30. The method of claim 29 wherein the received one or more analyte associated signals are wirelessly transmitted.

31. A kit, comprising:
an insertion device including:
a housing;
an insertion mechanism coupled to the housing; and
an introducer coupled to the insertion mechanism; and
an analyte sensor coupled to the introducer, the analyte sensor including an implantable portion with one or more electrodes for intradermal positioning under a skin layer of a user;
wherein upon actuation of the insertion mechanism, the introducer is configured pierce the skin layer of the user to transcutaneously position the implantable portion of the analyte sensor such that at least one of the one or more electrodes is in fluid contact with the dermal fluid of the user; and
further
wherein the introducer is removed from the user after positioning the implantable portion of the analyte sensor.

32. The kit of claim 31 wherein the insertion mechanism is spring biased.

33. The kit of claim 31 wherein the insertion mechanism is configured to automatically retract the introducer from the user after positioning the implantable portion of the analyte sensor in the dermal layer of the user.

34. The kit of claim 31 wherein the insertion mechanism is configured to position the analyte sensor at a predetermined angle relative to the skin layer.

35. The kit of claim 34 wherein the predetermined angle is less than or equal to approximately 180 degrees.

36. The kit of claim 31 wherein the analyte sensor is positioned substantially parallel to the skin layer.

37. The kit of claim 31 wherein the portion of the analyte sensor is transcutaneously positioned under skin layer such that a penetration depth of the introducer and the sensor is maintained above the subcutaneous layer of the user.

38. The kit of claim 31 wherein the one or more electrodes includes a working electrode.

39. The kit of claim 31 wherein the analyte sensor is configured to generate one or more signals associated with an analyte level of the user from the one or more electrodes in fluid contact with the dermal fluid.

40. The kit of claim 31 wherein the analyte sensor includes one or more of a glucose sensor or an oxygen sensor.

41. A method, comprising:
transcutaneously positioning an analyte sensor through a skin layer of a user, said analyte sensor including at least a working electrode, wherein said working electrode of the analyte sensor is positioned within a dermal layer under the skin layer; and
detecting a signal generated from the working electrode of the analyte sensor, wherein the signal is associated with an analyte level of the user,
wherein the generated signal from the working electrode of the analyte sensor is detected within a shorter time period from the sensor positioning, than a time period to detect a generated signal from a working electrode of a subcutaneous sensor having the working electrode positioned in the subcutaneous fluid measured from the same sensor positioning.

42. The method of claim 41 wherein transcutaneously positioning the analyte sensor includes positioning working electrode at a depth under the skin layer not exceeding approximately 3 millimeters.

43. The method of claim 41 wherein the working electrode of the analyte sensor is positioned within the dermal layer substantially in parallel to the skin layer.

44. The method of claim 41 wherein the working electrode of the analyte sensor is substantially in fluid contact with dermal fluid of the user.

45. The method of claim 41 wherein the analyte level includes glucose level.

46. The method of claim 41 wherein transcutaneous positioning the analyte sensor includes manually penetrating the skin layer.

47. The method of claim 41 wherein the working electrode of the analyte sensor is positioned under the skin layer at a depth not exceeding approximately 2 millimeters.

48. The method of claim 41 wherein working electrode of the analyte sensor is positioned under the skin layer at a depth not exceeding approximately 1 millimeter.
